(19) United States
(12) Patent Application Publication
(10) Pub. No.: US 2017/0304358 A1
(43) Pub. Date: Oct. 26, 2017

(54) PRIMARY AND SECONDARY PREVENTION OF DEMENTIAS AND SUICIDE WITH TRACE DOSE LITHIUM

(71) Applicant: Seyyed Nassir GHAEMI, Arlington, MA (US)

(72) Inventor: Seyyed Nassir GHAEMI, Arlington, MA (US)

(21) Appl. No.: 15/517,381

(22) PCT Filed: Oct. 6, 2015
(86) PCT No.: PCT/US2015/054267

§ 371 Date: Apr. 6, 2017

Related U.S. Application Data
(60) Provisional application No. 62/060,106, filed on Oct. 6, 2014.

(51) Int. Cl. A61K 33/14; A61K 31/28; A61K 33/24; A61K 33/00

(52) U.S. Cl. CPC A61K 33/14; A61K 33/24; A61K 31/28; A61K 33/00; A61K 2300/00

(57) ABSTRACT

The present invention relates to use of very low dose lithium for primary and secondary prevention of dementia-related disorders and suicide.

### RELATED APPLICATIONS

The present invention claims the benefit of priority of U.S. Provisional Application No. 62/060,106, filed Oct. 6, 2014, the contents of which are hereby incorporated by reference in their entirety.

### FIELD OF THE INVENTION

The present invention provides compositions and methods for treating dementias and suicide with trace doses of lithium. More specifically, the present invention provides compositions and methods for treating and preventing dementias associated with chronic traumatic encephalopathy (CTE), as well as suicide.

### BACKGROUND OF THE INVENTION

Mood disorders have a significant social and economic impact on our society that includes functional impairment, disability and/or loss of work productivity, and increased demand on health services. Over the past 40-60 years, there has been an increasing awareness of the social and economic impact of mood disorders such as, for example, bipolar disorder. Historically, mood disorders such as bipolar disorder have typically been treated with lithium. For example, lithium has been used mainly to treat bipolar disorder in standard medical practice at standard doses of about 900 mg/d (e.g., a range of 600-1200 mg/d, used to achieve blood levels of 0.6-1.0 ng/dl). Unfortunately, such standard doses of lithium typically have adverse side effects including renal toxicity. Relatively little is known about the disease modalities that underlie dementia and/or suicide. Accordingly, there is an urgent need in the art to identify compositions and methods that may be used to treat and/or prevent dementia and/or suicide.

### SUMMARY OF INVENTION

The present invention features compositions and methods for treating and preventing subjects suffering from, or at risk of suffering from, dementia-related disorders and/or suicide using low dose formulations of lithium.

In one aspect the invention provides a method of treating and/or preventing dementia in a subject suffering from, or at risk of suffering from, dementia, including the steps of determining the subject is suffering from, or at risk of suffering, from dementia; and administering to the subject a very low dose of lithium, or a salt thereof.

In an embodiment, the very low dose of lithium is selected from the group or ranges consisting of 5-100 mg/d, 5-50 mg/d, 5-25 mg/d, 5-20 mg/d, 5-15 mg/d, and 5-10 mg/d. In an embodiment, the very low dose of lithium is 5-25 mg/d. In an embodiment, the very low dose of lithium is selected from the group of ranges consisting of 1-100 mg/d, 1-50 mg/d, 1-25 mg/d, 1-20 mg/d, 1-15 mg/d, 1-10 mg/d range, and 1-5 mg/d. In an embodiment, the very low dose of lithium is 1-5 mg/d.

In an embodiment, the dementia is selected from the group consisting of chronic traumatic encephalopathy (CTE), Alzheimer's type, vascular dementia, Lewy Body dementia, Pick's disease, frontal dementia, white matter dementia and Binswanger's disease, senility, mixed dementia, HIV-related dementia, mild cognitive impairment (MCI), Creutzfeld-Jakob disease, Korsakoff's syndrome, progressive hydrocephalus, neurosyphilis, motor neuron disease, and amyotrophic lateral sclerosis. In an embodiment, the dementia is associated with CTE.

In an embodiment, determining further comprises a subject that has one or more factors selected from the group including presence of any potential future exposure to risk factors for any dementias (e.g., for Alzheimer's dementia: entering late middle age or early old age, namely the ages of 50-70 years; for vascular dementia: current or past diagnosis of hypertension or diabetes; for HIV-related dementia: being diagnosed with HIV infection), presence of multiple risk factors for the above conditions (e.g., for Alzheimer's dementia: presence of mood disorders along with entering late middle age; for vascular dementia: current or past diagnosis of hypertension and/or diabetes, with white matter changes on head MRI, with or without any evidence of cognitive impairment; for HIV-related dementia: being diagnosed with HIV and evidence of white matter abnormalities on MRI).

In an embodiment, the lithium salt is selected from the group consisting of lithium carbonate, lithium citrate, lithium sulfate, lithium chloride. In an embodiment, the lithium salt is lithium citrate. In an embodiment, the subject is 50-70 years of age.

In an embodiment, the treatment further comprises simultaneously treating subjects suffering from, or at risk of suffering from, suicidal thoughts.

In another aspect, the invention provides for a method of treating and/or preventing suicidal thoughts in a subject suffering from, or at risk of suffering from, suicidal thoughts, including the steps of determining the subject is suffering from, or at risk of suffering, from suicidal thoughts; and administering to the subject a very low dose of lithium, or a salt thereof.

In an embodiment, the very low dose of lithium is selected from the group or ranges consisting of 5-100 mg/d, 5-50 mg/d, 5-25 mg/d, 5-20 mg/d, 5-15 mg/d, and 5-10 mg/d. In an embodiment, the very low dose of lithium is 5-25 mg/d. In an embodiment, the very low dose of lithium is selected from the group of ranges consisting of 1-100 mg/d, 1-50 mg/d, 1-25 mg/d, 1-20 mg/d, 1-15 mg/d, 1-10 mg/d range, and 1-5 mg/d. In an embodiment, the very low dose of lithium is 1-5 mg/d.

In an embodiment, the suicidal thoughts further comprises suicidal thoughts selected from the group consisting of suicide attempts, parasuicidal thoughts, parasuicidal behavior, post-traumatic stress disorder, chronic traumatic encephalopathy (CTE) and traumatic brain injury. In an embodiment, the dementia is CTE.

In an embodiment, determining further includes a subject that has one or more factors selected from the group including past trauma or abuse (sexual or physical or emotional), anger or rage, medical illnesses (like chronic pain syndrome or HIV-related illnesses or terminal diseases like end-stage cancer) or end-organ damage like severe liver or kidney disease, or major life stresses like losing a job or financial distress or grief upon the death of a loved one or loss of relationship, effects of bullying, social isolation, and the influence of others who have committed suicide (emotional contagion, folie a deux and/or influences spread by the media such as social media, television, movies or the news), past suicide attempts, family history of suicide or suicide attempts, presence of mood illnesses (bipolar disorder or major depressive disorder), anxiety conditions like post-traumatic stress disorder (PTSD) or obsessive compulsive disorder (OCD) or panic disorder, psychotic illnesses like schizophrenia or schizoaffective disorders, and personality disorders (like borderline or antisocial).

In an embodiment, the lithium salt is selected from the group consisting of lithium carbonate, lithium citrate, lithium sulfate, lithium chloride. In an embodiment, the lithium salt is lithium citrate.

In an embodiment, the subject is 50-70 years of age.

In an embodiment, the treatment further comprises simultaneously treating subjects suffering from or at risk of suffering from dementia.

In another aspect, the invention provides for a method of treating and/or preventing chronic traumatic encephalopathy (CTE) in a subject suffering from, or at risk of suffering from, chronic traumatic encephalopathy (CTE), including the steps of determining the subject is suffering from, or at risk of suffering, from chronic traumatic encephalopathy (CTE); and administering to the subject a very low dose of lithium, or a salt thereof.

In an embodiment, determining further comprises a subject has one or more factors selected from the group including participation in contact sports, stationed in military combat with likely exposure to projectiles; and/or subject has sustained one or more concussive or sub-concussive injuries to the head with or without CTE-related clinical mood/cognitive/behavioral symptoms.

In an embodiment, the lithium is lithium carbonate.

In an embodiment, the very low dose of lithium carbonate is selected from the group of ranges consisting of 5-100 mg/d, 5-50 mg/d, 5-25 mg/d, 5-20 mg/d, 5-15 mg/d, and 5-10 mg/d.

In an embodiment, the very low dose of lithium carbonate is 5-25 mg/d.

In an embodiment, the very low dose of lithium carbonate is selected from the group of ranges consisting of 1-100 mg/d, 1-50 mg/d, 1-25 mg/d, 1-20 mg/d, 1-15 mg/d, 1-10 mg/d range, and 1-5 mg/d.

In an embodiment, the very low dose of lithium carbonate is 1-5 mg/d.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

As used herein, “administering” refers to local and/or systemic administration, e.g., including enteral and parenteral administration. Routes of administration for the active agent(s) described herein include, but are not limited to oral administration, administration as a suppository, topical contact, intravenous administration, intraperitoneal administration, intramuscular administration, intralesional administration, nasal administration, subcutaneous administration, the implantation of a slow-release and/or regulated release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, ionophoretic and intracranial. Other modes of delivery include, but are not limited to, the use of liposomal formulations, intravenous infusion, transdermal patches, etc. In certain embodiments administering can include causing to be administered for example by prescribing for a subject, annotating a patient's medical record, instructing an agent or other person to administer, and the like.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

“Detect” refers to identifying the presence, absence or amount of a substance to be detected.

By “diagnostic” is meant any method that identifies the presence of a pathologic condition or characterizes the nature of a pathologic condition (e.g., a psychological condition such as, e.g., a mood disorder or dementia). Diagnostic methods differ in their sensitivity and specificity. While a particular diagnostic method may not provide a definitive diagnosis of a condition, it suffices if the method provides a positive indication that aids in diagnosis.

The phrase “effective amount” means a dosage or dosage regimen sufficient to produce a desired result (e.g., a lithium dose).

The term “lithium” as used herein without the descriptors “impurities” or “ions”, refers to any form of elemental lithium in a pharmaceutical composition that is an active ingredient as known in the art of pharmacy; “lithium” can refer to a lithium compound, and/or lithium salts, and/or any type of lithium admixture capable of yielding lithium-ions as an active principle in subjects (e.g. lithium aspartate, lithium carbonate, lithium citrate, lithium gluconate, lithium orotate, NPO3—which is a low-dose lithium microemulsion, any combinations thereof, and the like).

By “sensitivity” is meant the percentage of subjects with a particular disease (e.g., a mood disorder, a dementia, a dementia associated with chronic traumatic encephalopathy, and the like) that are correctly detected as having the disease.

By “specificity” is meant the percentage of subjects without a particular disease who test negative.

By “reduces” is meant a negative alteration of at least 10%, 25%, 50%, 75%, or 100%.

By “reference” is meant a standard or control condition.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments the subject may not be under the care or prescription of a physician or other health worker.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “subtherapeutic dosage” refers to a dosage that is below that typically used for the subject agent in typical therapeutic or prophylactic use.

The terms “systemic administration” and “systemically administered” refer to a method of administering one or more compound(s) or composition(s) to a mammal so that the compound(s) or composition(s) are delivered to sites in the body, including the targeted site of pharmaceutical action, via, for example, the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE FIGURES

FIG. 1 shows a four by four table with the two populations and two types of prevention described and the proposed benefits to be seen with very low dose lithium and its expected dosage range.

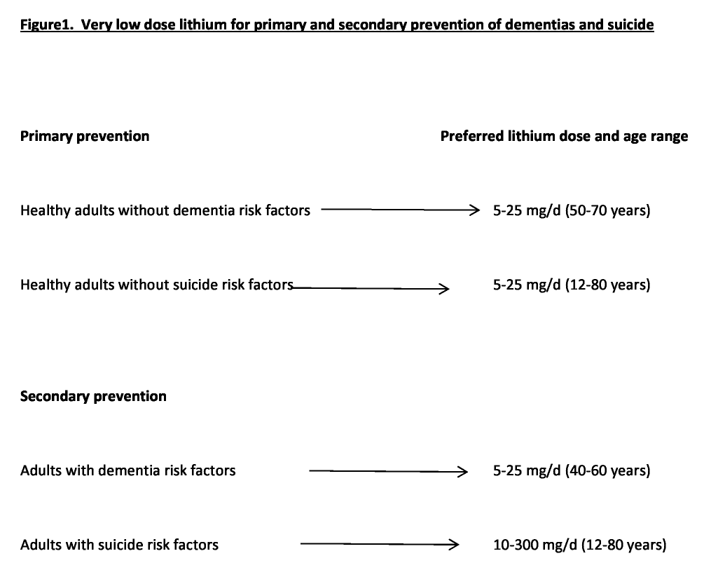

### DETAILED DESCRIPTION OF THE INVENTION

The invention features compositions and methods that are useful for treating and/or preventing dementia and/or suicide in a subject suffering from, or at risk of suffering from, dementia-related disorders and/or suicide using low or trace dose lithium. In particular, the present invention features compositions and methods useful for treating dementias associated with chronic traumatic encephalopathy (CTE). The present invention further features compositions and methods useful for treating dementias associated with mood disorders (e.g., bipolar disorder), and more particularly, for preventing future dementia in subjects aged 40-70 diagnosed with a mood disorder (e.g., bipolar disorder). The present invention is based, at least in part, on the inventor's discovery that trace lithium dosing in the range of 5-100 mg/d of lithium carbonate is effective in treating and/or preventing dementias and/or suicide in subjects suffering from, or prone to suffering from, dementia and/or suicide.

The present invention provides specific dosage recommendations by targeting doses to different age groups based on presence or absence of risk factors for dementias or suicide.

Human dementia studies indicate that standard doses of lithium have some benefit in older adults with minimal cognitive impairment (MCI), and one study indicated that “micro-doses” (<0.28 mg/d of lithium carbonate) had some benefit for cognition in persons with MCI. Animal studies indicate that lithium keeps neurons alive longer, which is consistent with a benefit for dementia prevention, even at very low doses of lithium (equivalent to blood levels of about 0.2 ng/dl or less, usually obtained with doses of about 300 mg/d of lithium carbonate in humans). According to the techniques herein, very low doses of lithium carbonate in the 1-300 mg/d range may have benefits in prevention of dementias or suicide. More specifically, very low doses of lithium carbonate in the 1-100 mg/d range, 1-50 mg/d range, 1-25 mg/d range, 1-20 mg/d range, 1-15 mg/d range, 1-10 mg/d range, 1-5 mg/d range may have benefits in prevention of dementias or suicide. Furthermore, very low doses of lithium carbonate in the 2-100 mg/d range, 2-50 mg/d range, 2-25 mg/d range, 2-20 mg/d range, 2-15 mg/d range, and 2-10 mg/d range may have benefits in prevention of dementias or suicide. Furthermore, very low doses of lithium carbonate in the 3-100 mg/d range, 3-50 mg/d range, 3-25 mg/d range, 3-20 mg/d range, 3-15 mg/d range, and 3-10 mg/d range may have benefits in prevention of dementias or suicide. Furthermore, very low doses of lithium carbonate in the 4-100 mg/d range, 4-50 mg/d range, 4-25 mg/d range, 4-20 mg/d range, 4-15 mg/d range, and 4-10 mg/d range may have benefits in prevention of dementias or suicide. Furthermore, very low doses of lithium carbonate in the 5-100 mg/d range, 5-50 mg/d range, 5-25 mg/d range, 5-20 mg/d range, 5-15 mg/d range, and 5-10 mg/d range may have benefits in prevention of dementias or suicide. It is contemplated within the scope of the invention that lithium carbonate administered in the range of 5-25 mg/d may be preferred for treating and/or preventing certain types of dementias and suicide. Moreover, it is specifically contemplated within the scope of the invention that all sub-ranges within the 5-25 mg/d range may be preferred for treating and/or preventing certain types of dementias and or suicide such as, for example, dementias associated with CTE.

This invention provides that very low dose lithium carbonate (e.g., 1-300 mg/d, 1-200 mg/d, 1-100 mg/d, 1-50 mg/d, 1-25 mg/d, 1-10 mg/d, 5-100 mg/d range, 5-50 mg/d range, 5-25 mg/d range, 5-20 mg/d range, 5-15 mg/d range, and 5-10 mg/d, and the like) may be used for primary and secondary prevention of dementias and suicide.

According to the techniques herein, for dementia prevention, primary prevention should begin at age 40-60 at a lithium carbonate dose of 1-5 mg/d, obtained by, for example, taking 1-2 drops of lithium citrate in water or another beverage daily. One of skill in the art will understand that higher doses of 10-100 mg/d may be given to persons with certain risk factors for dementias (e.g., genetic risk of Alzheimer dementia, mood illnesses like bipolar disorder and major depressive disorder, diabetes, hypertension, Parkinson's disease, Huntington's chorea, Down syndrome, and other neurodegenerative diseases). For suicide prevention, it is contemplated within the scope of the invention that lithium carbonate dosing of 1-5 mg/d may be desirable, while higher doses up to 300 mg/d may be desirable with respect to persons with major risk factors, including, but not limited to, mood illnesses (bipolar disorder or major depressive disorder), anxiety conditions like post-traumatic stress disorder (PTSD) or obsessive compulsive disorder (OCD) or panic disorder, psychotic illnesses like schizophrenia or schizoaffective disorders, and personality disorders (e.g., borderline or antisocial).

The invention provides specific dosing and population parameters for the use of very low, or trace, dose lithium in the primary and secondary treatment and/or prevention of dementias and suicide. According to one aspect of the invention, low dose or trace lithium, as described above, may be used for prevention in healthy persons without diagnosable dementia, and even in healthy persons with specific risk factors for suicide (i.e., primary prevention). Such risk factors may include, but are not limited to, family history of suicide or suicide attempts, anxiety symptoms (e.g., panic attacks or marked anxiety), alcohol or substance abuse, impulsivity or aggression, past trauma or abuse (e.g., sexual or physical or emotional) anger or rage, medical illnesses (e.g., chronic pain syndrome or HIV-related illnesses or terminal diseases like end-stage cancer) or end-organ damage (e.g., severe liver or kidney disease, or major life stresses like losing a job or financial distress or grief upon the death of a loved one or loss of relationship, effects of bullying, social media, television, movies or the news). This invention is also intended for treatment and/or prevention in persons who do not have diagnosable dementias or who have not yet committed suicide but who have risk factors for either condition (i.e., secondary prevention). Advantageously; in this circumstance, low or trace dose lithium use is primarily preventive in this secondary prevention setting as well. Besides these uses, very low dose lithium in the specific vehicles and doses and populations given here can be used as a treatment for individuals diagnosed with dementia as a means of preventing progression of the disease.

The current state of the art in medical practice does not recognize the utility of lithium for treating and/or preventing dementia or suicide. Lithium carbonate is used in standard medical practice primarily to treat bipolar disorder only at standard doses of about 600-1200 mg/d. While prior art patents exist for “micro-dose” lithium carbonate (defined as less than 0.28 mg/d) for treatment of Alzheimer's disease (see e.g., WO 2005102366), they do not address the utility of low dose lithium for treating and/or preventing dementias and suicide. Additionally, the above prior art relates to micro-doses (e.g., lower than the 1 mg and higher range of the present invention) of lithium carbonate for treatment only, not prevention, of only Alzheimer's disease, not including any other types of dementias. Prior art patents also exist for the treatment of neurodegenerative disorders with low-dose lithium (defined as 0.1-10 mg/d) (see e.g., WO 2012007387), but do not specify treating and/or preventing dementias generally, let alone treatment and/or prevention of suicide. Prior art patents exist for lithium combinations for various uses, including suicide prevention, but are limited to combination with other psychotropic medications (see e.g., WO 2005102366), not lithium use by itself, and do not relate specifically to very low-dose lithium carbonate (range provided is 25 mg/d to 2000 mg/d).

Diseases Treated with Lithium

In some embodiments, the present disclosure provides for methods of treating and/or preventing dementia and/or suicide with low doses of lithium compositions. Diseases treated with low dose lithium include but are not limited to: dementia, Alzheimer's disease, senility, vascular dementia, Lewy body dementia, frontotemporal dementia, Pick's disease, mixed dementia, HIV-related dementia, mild cognitive impairment (MCI), Creutzfeld-Jakob disease, Korsakoff's syndrome, progressive supranuclear palsy, Binswanger's disease, white matter dementia, Parkinson's disease, Huntington's disease, normal pressure hydrocephalus, neurosyphilis, motor neuron disease, and amyotrophic lateral sclerosis, suicide, suicidal ideation, suicidal thoughts, suicide attempts, parasuicidal thoughts (thoughts of self-harm or self-mutilation that are not likely to be fatal or without intent to cause death), parasuicidal behavior (self-mutilation or self-harm not intended to cause death, including but not limited to self-cutting, head-banging, self-burning with cigarettes or other hot objects, scratching oneself, swallowing foreign objects or solvents or pesticides or other harmful liquids or solids), post-traumatic stress disorder (PTSD), traumatic brain injury (TBI), and chronic traumatic encephalopathy (CTE).

### Dementia

Dementia, also known as senility, is a broad category of brain diseases that cause a long term and often gradual decrease in the ability to think and remember that is great enough to affect a person's daily functioning. Other common symptoms include emotional problems, problems with language, and a decrease in motivation. A person's consciousness is not affected. A dementia diagnosis requires a change from a person's usual mental functioning and a greater decline than one would expect due to aging. These diseases also have a significant effect on a person's caregivers.

The most common type of dementia is Alzheimer's disease, which makes up 50% to 70% of cases. Other common types include vascular dementia (25%), Lewy body dementia (15%), and frontotemporal dementia. Less common causes include Pick's disease, mixed dementia, HIV-related dementia, mild cognitive impairment (MCI), Creutzfeld-Jakob disease, Korsakoff's syndrome, progressive supranuclear palsy, Binswanger's disease, white matter dementia, Parkinson's disease, Huntington's disease, normal pressure hydrocephalus, neurosyphilis, motor neuron disease, and amyotrophic lateral sclerosis. More than one type of dementia may exist in the same person. A small proportion of cases run in families. In the DSM-5, dementia was reclassified as a neurocognitive disorder, with various degrees of severity. Diagnosis is usually based on history of the illness and cognitive testing with medical imaging and blood work used to rule out other possible causes. The mini-mental state examination is one commonly used cognitive test. Efforts to prevent dementia include trying to decrease risk factors such as high blood pressure, smoking, diabetes and obesity. Screening the general population for the disease is not recommended.

There is no cure for dementia. Cholinesterase inhibitors such as donepezil are often used and may be beneficial in mild to moderate disease; however, the overall benefit is believed to be minor. For people with dementia and those who care for them many measures can improve their lives. Cognitive and behavioral interventions may be appropriate. Educating and providing emotional support to the caregiver is important. Exercise programs are beneficial with respect to activities of daily living and potentially improve outcomes. Treatment of behavioral problems or psychosis due to dementia with antipsychotics is common but not usually recommended due to there often being little benefit and an increased risk of death.

Dementia affects the brain's ability to think, reason, and remember clearly. The most commonly affected areas include memory, visual-spatial, language, attention, and executive function (i.e., problem solving). Most types of dementia are slow and progressive. By the time the person shows signs of the disease, the process in the brain has been happening for a long time. It is possible for a patient to have two types of dementia at the same time. About 10% of people with dementia have what is known as mixed dementia, which is usually a combination of Alzheimer's disease and another type of dementia such as, for example, frontotemporal dementia or vascular dementia. Additional psychological and behavioral problems that often affect people who have dementia include: balance problems, tremor, speech and language difficulty, trouble eating or swallowing, memory distortions (e.g., believing that a memory has already happened when it has not, thinking an old memory is a new one, combining two memories, or confusing the people in a memory), wandering or restlessness, behavioral and psychological symptoms of dementia (BPSD) almost always occur in all types of dementia. BPSDs may manifest as: agitation, depression, anxiety, abnormal motor behavior, elated mood, irritability, apathy, disinhibition and impulsivity, delusions (often believing people are stealing from them) or hallucinations, changes in sleep or appetite.

Depression and mania increase the risk of dementias up to four-fold and are major risk factors. Depression affects 20-30% of people who have dementia, and about 20% have anxiety. Psychosis (often delusions of persecution) and agitation/aggression also often accompany dementia. Each of these must be assessed and treated independently of the underlying dementia.

### Suicide

Suicide is the act of intentionally causing one's own death. Suicide is often carried out as a result of despair, the cause of which is frequently attributed to a mental disorder such as depression, bipolar disorder, schizophrenia, borderline personality disorder, alcoholism, or drug abuse, as well as risk factors such as past trauma or abuse (sexual or physical or emotional), anger or rage, medical illnesses (like chronic pain syndrome or HIV-related illnesses or terminal diseases like end-stage cancer) or end-organ damage like severe liver or kidney disease, or major life stresses like losing a job or financial distress or grief upon the death of a loved one or loss of relationship, effects of bullying, social isolation, and the influence of others who have committed suicide (emotional contagion, folie a deux and/or influences spread by the media such as social media, television, movies or the news). Suicide prevention efforts include limiting access to method of suicide such as firearms and poisons, treating mental illness and drug misuse, and improving economic circumstances.

Suicide, also known as completed suicide, is the “act of taking one's own life.” Attempted suicide or non-fatal suicidal behavior is self-injury with the desire to end one's life that does not result in death. Assisted suicide is when one individual helps another bring about their own death indirectly via providing either advice or the means to the end. This is in contrast to euthanasia, where another person takes a more active role in bringing about a person's death. Suicidal ideations are thoughts of ending one's life but not taking any active efforts to do so.

Mental disorders are often present at the time of suicide with estimates ranging from 27% to more than 90%. Of those who have been admitted to a psychiatric unit, their lifetime risk of completed suicide is about 8.6%. Half of all people who die by suicide may have major depressive disorder; having this or one of the other mood disorders such as bipolar disorder increases the risk of suicide 20-fold. Other conditions implicated include schizophrenia (14%), personality disorders (14%), bipolar disorder, and posttraumatic stress disorder. About 5% of people with schizophrenia die of suicide. Eating disorders are another high risk condition.

A history of previous suicide attempts is the greatest predictor of eventual completion of suicide. Approximately 20% of suicides have had a previous attempt, and of those who have attempted suicide, 1% complete suicide within a year and more than 5% commit suicide within 10 years. Acts of self-harm are not usually suicide attempts and most that self-harm are not at high risk of suicide. Some who self-harm, however, do still end their life by suicide, and risk for self-harm and suicide may overlap.

In approximately 80% of completed suicides the individual has seen a physician within the year before their death, including 45% within the prior month. Approximately 25-40% of those who completed suicide had contact with mental health services in the prior year.

### Chronic Traumatic Encephalopathy (CTE)

Chronic traumatic encephalopathy (CTE) is a form of encephalopathy that is a progressive degenerative disease, which is currently only definitively diagnosed on autopsy. Baseline testing has been created to assess potential cognitive impairment in athletes in contact sports, but a test to determine the presence of CTE while the person is alive and a conventional postmortem diagnosis is not yet available. The disease was previously called dementia pugilistica (DP), i.e. “punch-drunk”, as it was initially found in those with a history of boxing. CTE has been most commonly found in professional athletes participating in American football, association football, soccer, ice hockey, professional wrestling, boxing, mixed martial arts, rugby, baseball, Australian football, and other contact sports that have experienced repetitive brain trauma. It has also been found in soldiers exposed to a blast or a concussive injury, in both cases resulting in characteristic degeneration of brain tissue and the accumulation of tau protein. Individuals with CTE may show symptoms of dementia, such as memory loss, aggression, confusion and depression, which may appear years or many decades after the trauma.

CTE is a progressive degenerative disease of the brain found in people with a history of repetitive brain trauma, including symptomatic concussions as well as sub-concussive hits to the head that do not cause symptoms. In the case of blast injury, a single exposure to a blast and the subsequent violent movement of the head in the blast wind can cause the condition.

The only definitive diagnosis of CTE can be made post-mortem with the study of the brain, but some biomarkers are being studied now. Clinical symptoms are in three categories: mood (depression, apathy, irritability, mood lability, anxiety), cognition (word-finding difficulty, executive function impairment, inattention, poor working and short-term memory, and eventually poor long-term memory), and behavior (aggression toward others and toward oneself, sometimes suicide and even homicide). MRI evaluation can demonstrate evidence of brain atrophy, both cortical and subcortical, with ventricular enlargement, sulcar enlargement, and white matter changes. Post-mortem the classic pattern of diffuse tau deposition is found in dense neurofibrillary tangles; beta-amyloid deposition is uncommon, in contrast to Alzheimer's dementia. Early studies now suggest the possible ability to detect tau deposition by PET imaging.

Risk factors involve the experience of any concussion or sub-concussive head injury while playing any of the contact sports listed above, or by means of other head injury, such as being exposed as a member of the military to bombs or other projectiles. Other risk factors are epilepsy, being the victim of domestic abuse, being young in age at earliest exposure, length of sport participation, and possibly genetic variations of the ApoE3 or E4 genes.

### Lithium

### Compositions

In the present invention, lithium compositions such as lithium carbonate may be used to treat (or prevent) dementia-related disorders and/or suicide at doses less than 300 mg/d in humans. In some embodiments, doses are about 1-300 mg/d in humans. In other embodiments, doses of lithium are administered at about 1-5 mg/d in humans. In other embodiments, very low doses of lithium in the about 1-100 mg/d range, 1-50 mg/d range, 1-25 mg/d range, 1-20 mg/d range, 1-15 mg/d range, 1-10 mg/d range, 1-5 mg/d range may have benefits in prevention of dementias or suicide. In other embodiments, very low doses of lithium in the about 2-100 mg/d range, 2-50 mg/d range, 2-25 mg/d range, 2-20 mg/d range, 2-15 mg/d range, and 2-10 mg/d range may have benefits in prevention of dementias or suicide. In other embodiments, very low doses of lithium in the about 3-100 mg/d range, 3-50 mg/d range, 3-25 mg/d range, 3-20 mg/d range, 3-15 mg/d range, and 3-10 mg/d range may have benefits in prevention of dementias or suicide. In other embodiments, very low doses of lithium in the about 4-100 mg/d range, 4-50 mg/d range, 4-25 mg/d range, 4-20 mg/d range, 4-15 mg/d range, and 4-10 mg/d range may have benefits in prevention of dementias or suicide. In other embodiments, very low doses of lithium in the about 5-100 mg/d range, 5-50 mg/d range, 5-25 mg/d range, 5-20 mg/d range, 5-15 mg/d range, and 5-10 mg/d range may have benefits in prevention of dementias or suicide. It is contemplated within the scope of the invention that lithium administered in the range of about 5-25 mg/d may be preferred for treating and/or preventing certain types of dementias and suicide. In other embodiments, humans may receive lithium doses of less than 1 mg/d to treat and/or prevent dementia-related disorders and/or suicide.

While the exemplary dose ranges are given with respect to lithium carbonate, one of skill in the art will appreciate that the amount of elemental lithium may change depending on the particular salt. For example, there is about ⅕ the amount of elemental lithium for every mg of the carbonate salt (18.8 mg of elemental lithium per 100 mg of lithium carbonate). Similarly, there is ⅕ the amount of elemental lithium in lithium orotate (3.83 mg/100 mg) as in lithium carbonate (18.8 mg/100 mg). It is contemplated within the scope of the invention that the very low dose of lithium derived from other salt forms may be quantitatively modified relative to the above-identified ranges for lithium carbonate to provide the same resulting amount of elemental lithium.

For example, dose ranges proposed for elemental lithium as derived from lithium carbonate would be in the following amounts: 1-5 mg/d of lithium carbonate would be equivalent to 0.2-1 mg/d of elemental lithium; 1-100 mg/d of lithium carbonate would be equivalent to 0.2-20 mg/d of elemental lithium; 1-300 mg/d of lithium carbonate would be equivalent to 0.2-60 mg/d of elemental lithium; 5-25 mg/d of lithium carbonate would be equivalent to 1-5 mg/d of elemental lithium.

The administration of a lithium composition according to the present invention may conveniently be parenteral or preferably oral (e.g., tablet, capsule, orally dissolvable table, liquid, a skin patch, and the like), and the compound, which is in a form from which lithium ions are biologically available, may be a salt such as the carbonate, citrate, succinate, chloride, bromide, acetate, acetylsalicylate, benzoate, bitartrate, nitrate, orotate, selenate, selenite, sulphate, aspartate, gluconate or thenoate.

Lithium is available in a wide number of pharmaceutical formulations. Typically, lithium is provided as a salt e.g., lithium carbonate (Li2CO3), lithium citrate (Li3C6H5O7), lithium sulfate (Li2SO4), lithium chloride (LiCl), and the like. Suitable lithium salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, orotate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

The lithium compositions comprising the formulations described herein of this invention are typically each combined with a separate or the same pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agent(s), or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the combined formulation(s) and on the particular physio-chemical characteristics of the active agent(s) comprising the formulation. In certain embodiments the excipients are preferably sterile and generally free of undesirable matter. These compositions may be sterilized by conventional, well-known sterilization techniques.

In general, the solubility of lithium salts of organic acids in water or aqueous solutions vary unpredictably. Lithium carbonate, for example, is slightly soluble in water and insoluble in ethyl alcohol. Lithium acetate is soluble in water and ethyl alcohol. Lithium citrate is soluble in water and slightly soluble in ethyl alcohol.

### Administration

Lithium formulations of the present invention may be administered in a number of ways to treat and/or prevent dementia-related disorders (e.g., CTE) and/or suicide. The lithium compositions of the invention are administered alone or co-administered to the patient. Co-administration is meant to include simultaneous or sequential administration of the compositions individually or in combination (more than one compound). Thus, the preparations are also combined, when desired, with other active substances.

The compositions of the invention are administered in a variety of routes including but not limited to: oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The compositions of the present invention are prepared and administered in a wide variety of oral, parenteral and topical dosage forms. Oral preparations include tablets, orally dissolvable tablets, pills, powder, dragees, capsules, liquids, lozenges, cachets, gels, syrups, slurries, suspensions, etc., suitable for ingestion by the patient. The salts of the present invention may also administered by injection, that is, intravenously, intramuscularly, intracutaneously, subcutaneously, intraduodenally, or intraperitoneally. Also, the salts described herein may be administered by inhalation, for example, intranasally. Additionally, the salts of the present invention may be administered transdermally. It is also envisioned that multiple routes of administration (e.g., intramuscular, oral, transdermal) are used to administer the salts of the invention. Accordingly, the present invention also provides pharmaceutical compositions comprising a pharmaceutically acceptable excipient and one or more salts of the invention.

The pharmaceutical preparation is preferably in unit dosage form. In such form the preparation is subdivided into unit doses containing appropriate quantities of the active component. The unit dosage form is a packaged preparation, the package containing discrete quantities of preparation, such as packeted tablets, capsules, and powders in vials or ampoules. Also, the unit dosage form is a capsule, tablet, cachet, or lozenge itself, or it is the appropriate number of any of these in packaged form.

Generally, lithium may be given in a single dose or multiple administrations, i.e., once, twice, three or more times daily over a period of time. The composition, if desired, also contains other compatible therapeutic agents. Multiple unit doses are administered within a 24 hour time period.

### Mode of Implementation

According to the techniques herein, lithium citrate given in a dose range of 1-50 mg/d, 1-25 mg/d, 1-10 mg/d, or 1-5 mg/d may be preferred embodiments for very low dose treatment and/or prevention of dementias including, but not limited to, CTE. Higher doses such as 5-300 mg/d (and even up to 1200 mg/d, reaching standard lithium levels of 0.6 to 1.2 ng/dl) may also be used. It is contemplated within the scope of the invention, that the same dose range may be used for treating and/or preventing suicide, although doses as high as 300-1200 mg/d could be needed in more severe cases.

The present invention may provide easy access to very low dose lithium through liquid lithium citrate, which has an advantage relative to standard pill-based doses of lithium carbonate that do not come in pill sizes smaller than 150 mg/d.

Very low dose lithium may be provided as one or two drops of lithium citrate mixed with water or any other beverage as provided below. Lithium citrate can be given as currently marketed as 300 mg (or 8 mEq) per 5 ml (one teaspoon). Higher doses up to 300 mg/d can be obtained by measuring equivalent amounts from a 5 ml dropper, which is equivalent to 300 mg/d. Thus 50 mg/d of lithium is obtained by taking slightly less than 1 ml of lithium citrate. About 20 drops are thought to constitute one ml of a solution (60 mg of lithium); thus each drop of lithium citrate is equivalent to 3 mg of lithium citrate. Thus 2-8 drops from a teaspoon of that strength of lithium citrate will provide 6-24 mg/d of lithium citrate. Given daily or every other day, this usage would provide the 5-25 mg/d range described below. In elderly persons (above age 70), lithium citrate could be given once or twice weekly at 2-8 drops daily for the same benefit (less frequent dosing is necessary due to decreased renal clearance of medication). 10 mg/d of lithium is obtained by taking about 3 drops of lithium citrate. 100 mg/d of lithium is obtained by taking slightly less than 2 ml of lithium citrate. Other formulations of lithium in any of its varieties (citrate, carbonate, orotate, and others described below) can be produced in a pharmacological vehicle (pill or transdermal or other) with these very low doses; if prepared and marketed those pills or other vehicles for very low dose lithium can be taken instead of lithium citrate liquid as described here. One of skill in the art will appreciate that liquid lithium citrate may be formulated at a concentration minimal to the very low dose schedules provided by the invention.

Other types of lithium salts—carbonate, gluconate, orotate, acetate, aspartate, chloride—may also be pharmacologically prepared in very low dose formats to provide the very low dose schedules as described herein. Lithium citrate may also be prepared pharmacologically in very low doses in pill form. Some of the above forms may also be given transdermally.

The following examples are put forth so as to provide those of ordinary skill in the art with a complete disclosure and description of how to make and use the compositions and methods of the invention, and are not intended to limit the scope of what the inventors regard as their invention.

### EXAMPLES

The present invention uses lithium as dosed below in two populations and for two types of prevention:

### Example 1: Primary Prevention of Dementia

In the general population (no specific risk factors for dementias or suicide), very low dose lithium may be given for primary prevention as follows:

Primary prevention of dementias: Very low dose lithium (e.g., 1-50 mg/d, 1-25 mg/d, 1-10 mg/d, 1-5 mg/d, 5-25 mg/d, and the like) may be given to reduce the risk of later occurrence of dementias of any kind (CTE, Alzheimer's type, vascular dementia, Lewy Body dementia, Pick's disease, frontal dementia, white matter dementia or Binswanger's disease). If tolerated, lithium dose may be increased up to 100 mg/d, or possibly higher, such as 300 mg/d or even up to 900-1200 mg/d (with standard blood levels of 0.6 to 1.2 ng/dl), which is a typical dose for bipolar disorder. This dosing should preferentially begin when a subject has reached the age of 50, 60, or 70, or in some cases the ages of 40 or 90 (in otherwise healthy individuals who want to preserve cognitive function into very old age).

### Example 2: Primary Prevention of Suicide

Primary prevention of suicide: Very low dose lithium (e.g., 1-50 mg/d, 1-25 mg/d, 1-10 mg/d, 1-5 mg/d, 5-25 mg/d, and the like) may be given to reduce the risk of later occurrence of suicide in persons without prior psychiatric diagnoses of any kind. If tolerated, lithium dose may be increased up to 100 mg/d, or possible higher, such as 300 mg/d or even up to 900-1200 mg/d, which is a typical dose for bipolar disorder (with standard blood levels of 0.6 to 1.2 ng/dl). This dosing may begin in the decades of the teens or twenties or anytime thereafter.

### Example 3: Secondary Prevention of Dementia

In the population of persons with risk factors for dementias or suicide, very low dose lithium would be given for secondary prevention as follows:

Secondary prevention of dementias: Individuals may have risk factors for dementias, including a family history of dementias of any of the types listed below: CTE, minimal cognitive impairment syndrome (MCI); another major risk factor is current or past depression or any mood illness (i.e., major depressive disorder or bipolar disorder and their variants, such as dysthymia, cyclothymia, hyperthymia); other risk factors are current or past hypertension, diabetes, and other neurodegenerative or other neurological diseases such as Parkinson's disease, stroke, multiple sclerosis, Huntington's disease, epilepsy. These diagnoses are sufficient by themselves to be risk factors for dementias, but if they are combined with any cognitive impairment symptoms at all, those patients would be even higher risk subjects and would benefit from secondary prevention of dementias with very low dose lithium. In these persons with risk factors for dementias, very low dose lithium (e.g., 1-50 mg/d, 1-25 mg/d, 1-10 mg/d, 1-5 mg/d, 5-25 mg/d, and the like) may be given to reduce the risk of later occurrence of dementias of any kind (CTE, Alzheimer's type, vascular dementia, Lewy Body dementia, Pick's disease, frontal dementia, white matter dementia or Binswanger's disease). If tolerated, lithium dose can be increased up to 100 mg/d, or possibly higher in CTE, such as 300 mg/d or even up to 900-1200 mg/d, which is a typical dose for bipolar disorder (with standard blood levels of 0.6 to 1.2 ng/dl). This dosing should preferentially begin when a subject has reached the age of 50, 60, or 70, or in some cases the ages of 10 (for prevention of CTE or other early onset dementias like Down syndrome or Huntington's disease) or 90.

### Example 4: Secondary Prevention of Suicide

Secondary prevention of suicide: Individuals may have risk factors for suicide, most commonly psychiatric diagnoses of any kind, but especially any mood condition (depression, major depressive disorder, bipolar disorder, and their variants, including dysthymia, cyclothymia, hyperthymia); any anxiety condition (especially panic disorder or panic attacks, obsessive-compulsive disorder, post-traumatic stress disorder, and generalized anxiety disorder); any substance abuse condition (especially alcohol and illegal drug abuse or prescription opiate abuse); and any personality disorder (especially borderline personality disorder and antisocial personality disorder). Persons with past suicide attempts, past self-harm (parasuicide: self-cutting or other self-injury) are also at higher risk. Persons with a family history of any relative (first, second, or third degree) who committed suicide or made a suicide attempt is also at higher risk. Persons with CTE or other neurological disorders (e.g., multiple sclerosis, Parkinson's disease, epilepsy) also are at higher risk of suicide. In these persons with risk factors for suicide, very low dose lithium (1-5 mg/d) can be given to reduce the risk of later occurrence of suicide. If tolerated, lithium dose can be increased up to 300 mg/d. This dosing can begin in the decades of the teens or twenties or anytime thereafter. In persons with full diagnosed mood disorders (MDD or bipolar disorder), higher standard doses (600-1500 mg/d) and levels of lithium (0.6-1.2 ng/dl) may be needed and tolerated for mood episode prevention, and will also be useful for suicide prevention. In some such mood disorder patients, if higher standard doses are not needed for mood episode prevention, the low doses described here (1-5 mg/d, and increased if needed and tolerated up to 300 mg/d) will be effective for suicide prevention.

Table 1 details a summary of dosing and populations from Examples 1-4 according to one exemplary embodiment.

TABLE 1
SUMMARY OF DOSING AND POPULATIONS (lithium carbonate)

| | | | | Optimal | Extended |
|---|---|---|---|---|---|
| | | | | age range | age range |
| | | Preferred | Dose | for use | for use |
| Population | Purpose | dose | range | (years) | (years) |
| Healthy adult | Primary | 5-25 mg/d | 1-100 | 50-70 | 40-90 |
| without | Prevention of | | mg/d | | |
| dementia risk | dementias | | | | |
| factors | | | | | |
| Adults with | Secondary | 5-25 mg/d | 1-300 | 40-60 | 10-90 |
| dementia risk | prevention of | | mg/d | | |
| factors | dementias | | | | |
| Healthy adults | Primary | 5-25 mg/d | 1-100 | 12-80 | NA |
| without suicide | prevention of | | mg/d | | |
| risk factors | suicide | | | | |
| Adults with | Secondary | 10-300 | 1-300* | 12-80 | NA |
| suicide risk | prevention of | mg/d | mg/d | mg/d | |
| factors | suicide | | | | |

*Higher doses including standard lithium doses (600-1500 mg/d) and levels (0.6-1.2) can be used in persons diagnosed with MDD or bipolar mood disorders. The lower doses provided here refer either to persons with those diagnoses who cannot tolerate or do not need higher doses, or to persons with other psychiatric diagnoses or other reasons to be at-risk (e.g., CTE, family history of suicide or suicide attempts).

### Exemplary Embodiment for Treatment or Prevention of CTE

In exemplary embodiment related to treatment or prevention of CTE, lithium carbonate dose equivalents could be used in the following manner: For primary prevention in young children or adolescents or adults who are entering sports activities that involve possible concussive or sub-concussive impacts on the head, lithium carbonate could be administered to prevent harmful impact of such head trauma in preferred doses of 5-25 mg/d (range 1-100 mg/d). In recruits into the military being sent to regions of warfare with possible projectile-related head injury, lithium carbonate could be given as primary prevention in preferred doses of 5-25 mg/d (range 1-100 mg/d). In persons who have experienced at least one concussion or sub-concussive impact in sports activities and/or military experience, with or without prior use of lithium, the agent could be given in secondary prevention in preferred doses of 25-100 mg/d (range 5-300 mg/d). In persons who have experienced at repeated concussion or sub-concussive impacts in sports activities and/or military experience, with or without prior use of lithium, the agent could be given in secondary prevention in preferred doses of 100-300 mg/d (range 25-600 mg/d), depending on clinical presentation. For instance, if no mood or cognitive or behavioral symptoms are present clinically, the lower range of the above doses could be used (25-100 mg/d); if some mood changes are present (like depression and/or anxiety and/or irritability) but no other cognitive or behavioral changes, moderate doses could be given (100-300 mg/d); if both mood and cognition is affected but aggressive behavior is not evident, higher doses could be given but still below “standard” doses used in bipolar disorder (300-600 mg/d); if full CTE-like symptoms are present clinically (mood, cognitive and behavioral changes with aggression and impulsivity), “standard” doses can be given similar to those used in bipolar disorder (600-1200 mg/d, with standard blood levels of 0.6 to 1.2 ng/dl). If suicidality is present at any point in the clinical presentation, doses might be increased from prior amounts (for example, very low doses of 5-25 mg/d would be increased to the 100-300 mg/d range).

### REFERENCES

1. S Mauer, D Vergne, S N Ghaemi. Standard and trace-dose lithium: A systematic review of dementia prevention and other behavioral benefits. Aust N Z J Psychiatry. 2014 September; 48(9):809-818.

1. A method of treating and/or preventing dementia in a subject suffering from, or at risk of suffering from, dementia, comprising:
determining the subject is suffering from, or at risk of suffering, from dementia; and
administering to the subject a very low dose of lithium, or a salt thereof.

2. The method of claim 1, wherein the very low dose of lithium is selected from the group of ranges consisting of 5-100 mg/d, 5-50 mg/d, 5-25 mg/d, 5-20 mg/d, 5-15 mg/d, and 5-10 mg/d.

3. The method of claim 2, wherein the very low dose of lithium is 5-25 mg/d.

4. The method of claim 1, wherein the very low dose of lithium is selected from the group of ranges consisting of 1-100 mg/d, 1-50 mg/d, 1-25 mg/d, 1-20 mg/d, 1-15 mg/d, 1-10 mg/d range, and 1-5 mg/d, or the very low dose of lithium carbonate is 1-5 mg/d.

5. (canceled)

6. The method of claim 1, wherein the dementia is selected from the group consisting of chronic traumatic encephalopathy (CTE), Alzheimer's type, vascular dementia, Lewy Body dementia, Pick's disease, frontal dementia, white matter dementia and Binswanger's disease, senility, mixed dementia, HIV-related dementia, mild cognitive impairment (MCI), Creutzfeld-Jakob disease, Korsakoff's syndrome, progressive hydrocephalus, neurosyphilis, motor neuron disease, and amyotrophic lateral sclerosis, or the dementia is CTE.

7. (canceled)

8. The method of claim 1, wherein determining further comprises a subject has one or more factors selected from the group consisting of: presence of any potential future exposure to risk factors for any dementias (e.g., for Alzheimer's dementia: entering late middle age or early old age, namely the ages of 50-70 years; for vascular dementia: current or past diagnosis of hypertension or diabetes; for HIV-related dementia: being diagnosed with HIV infection), presence of multiple risk factors for the above conditions (e.g., for Alzheimer's dementia: presence of mood disorders along with entering late middle age; for vascular dementia: current or past diagnosis of hypertension and/or diabetes, with white matter changes on head MRI, with or without any evidence of cognitive impairment; for HIV-related dementia: being diagnosed with HIV and evidence of white matter abnormalities on MRI).

9. The method of claim 1, wherein the lithium salt is selected from the group consisting of lithium carbonate, lithium citrate, lithium sulfate, lithium chloride.

10. (canceled)

11. The method of claim 1, wherein the subject is 50-70 years of age.

12. The method of claim 1, wherein the treatment further comprises simultaneously treating subjects suffering from or at risk of suffering from suicidal thoughts.

13. A method of treating and/or preventing suicidal thoughts in a subject suffering from, or at risk of suffering from, suicidal thoughts, comprising:
determining the subject is suffering from, or at risk of suffering, from suicidal thoughts; and
administering to the subject a very low dose of lithium, or a salt thereof.

14-15. (canceled)

16. The method of claim 13, wherein the very low dose of lithium is selected from the group of ranges consisting of 1-100 mg/d, 1-50 mg/d, 1-25 mg/d, 1-20 mg/d, 1-15 mg/d, 1-10 mg/d range, and 1-5 mg/d, or the very low dose of lithium is 1-5 mg/d.

17. (canceled)

18. The method of claim 13, wherein the suicidal thoughts further comprises suicidal thoughts selected from the group consisting of suicide attempts, parasuicidal thoughts, parasuicidal behavior, post-traumatic stress disorder, chronic traumatic encephalopathy (CTE) and traumatic brain injury.

19. (canceled)

20. The method of claim 13, wherein determining further comprises a subject has one or more factors selected from the group consisting of: past trauma or abuse (sexual or physical or emotional), anger or rage, medical illnesses (like chronic pain syndrome or HIV-related illnesses or terminal diseases like end-stage cancer) or end-organ damage like severe liver or kidney disease, or major life stresses like losing a job or financial distress or grief upon the death of a loved one or loss of relationship, effects of bullying, social isolation, and the influence of others who have committed suicide (emotional contagion, folie a deux and/or influences spread by the media such as social media, television, movies or the news), past suicide attempts, family history of suicide or suicide attempts, presence of mood illnesses (bipolar disorder or major depressive disorder), anxiety conditions like post-traumatic stress disorder (PTSD) or obsessive compulsive disorder (OCD) or panic disorder, psychotic illnesses like schizophrenia or schizoaffective disorders, and personality disorders (like borderline or antisocial).

21. The method of claim 13, wherein the lithium salt is selected from the group consisting of lithium carbonate, lithium citrate, lithium sulfate, lithium chloride.

22. (canceled)

23. The method of claim 13, wherein the subject is 50-70 years of age.

24. The method of claim 13, wherein the treatment further comprises simultaneously treating subjects suffering from or at risk of suffering from dementia.

24. A method of treating and/or preventing chronic traumatic encephalopathy (CTE) in a subject suffering from, or at risk of suffering from, chronic traumatic encephalopathy (CTE), comprising:
determining the subject is suffering from, or at risk of suffering, from chronic traumatic encephalopathy (CTE); and
administering to the subject a very low dose of lithium, or a salt thereof.

25. The method of claim 24, wherein determining further comprises a subject has one or more factors selected from the group consisting of: participation in contact sports, stationed in military combat with likely exposure to projectiles; and/or subject has sustained one or more concussive or sub-concussive injuries to the head with or without CTE-related clinical mood/cognitive/behavioral symptoms.

26. The method of claim 25, wherein the lithium is lithium carbonate.

27-28. (canceled)

29. The method of claim 26, wherein the very low dose of lithium carbonate is selected from the group of ranges consisting of 1-100 mg/d, 1-50 mg/d, 1-25 mg/d, 1-20 mg/d, 1-15 mg/d, 1-10 mg/d range, and 1-5 mg/d, or the very low dose of lithium carbonate is 1-5 mg/d.

30. (canceled)
